(19) United States
(12) Patent Application Publication
(10) Pub. No.: US 2026/0325125 A1
(43) Pub. Date: Oct. 22, 2026

(54) SUTURE ANCHOR ASSEMBLY

(71) Applicant: Arthrex, Inc., Naples, FL (US)

(72) Inventors: Phillip CHEE, Bonita Springs, FL (US); Brittany FOSLIEN, Naples, FL (US)

(21) Appl. No.: 19/653,762

(22) Filed: Apr. 21, 2026

Related U.S. Application Data
(60) Provisional application No. 63/791,821, filed on Apr. 21, 2025.
(60) Provisional application No. 63/862,268, filed on Aug. 12, 2025.

(51) Int. Cl. A61B 17/04

(52) U.S. Cl. CPC A61B 17/0401; A61B 2017/0408; A61B 2017/0412; A61B 2017/044; A61B 2017/0453; A61B 2017/0464

(57) ABSTRACT

An anchor assembly secures a tissue to a bone with at least one repair strand. The anchor assembly includes an anchor tip, an anchor sleeve, and a drive shaft. The anchor tip has a channel extending along a longitudinal axis. The channel receives the at least one repair strand. The anchor sleeve is inserted into a bone socket of the bone. The anchor sleeve has an outer thread on an outer surface. The drive shaft pushes the anchor tip into the bone socket and rotates the anchor sleeve to secure the at least one repair strand between the outer surface of the anchor sleeve and the bone socket.

### CROSS REFERENCE TO RELATED APPLICATIONS

This application claims the benefit of U.S. Provisional Patent App. No. 63/791,821, filed Apr. 21, 2025, and the benefit of U.S. Provisional Patent App. No. 63/862,268, filed Aug. 12, 2025, the disclosures of which are hereby incorporated by reference herein.

### TECHNICAL FIELD

The disclosure relates to the field of surgery and, more specifically, to anchors and methods of anchoring sutures.

### BACKGROUND

Repair or reattachment of tissue to bone is often performed by suturing. The procedure typically requires the surgeon to pass suture material through the tissue and to anchor the suture to the bone. However, knot tying during surgery can be tedious and time-consuming. Knots have a tendency to deform or collapse as the surgeon manually forces the knots down into the proper position. The knots and sutures themselves are also often exposed to abrasion or cutting by rough areas along walls of a bone canal into which anchors are typically inserted to provide fixation of tendon to bone.

### SUMMARY

Accordingly, a need exists for an improved method for attaching a tissue to a bone which facilitates tensioning of the suture when initially inserted and which allows the tissue to remain securely in place without multiple suture knots.

Thus, a first aspect of the present disclosure is directed to an anchor assembly configured to secure a tissue to a bone using at least one repair strand. The anchor assembly comprises an anchor tip having a channel extending along a longitudinal axis. The channel is configured to receive the at least one repair strand. The anchor assembly also comprises an anchor sleeve configured to be inserted into a bone socket of the bone. The anchor sleeve has an outer thread on an outer surface. The anchor assembly also comprises a drive shaft configured to push the anchor tip into the bone socket and to rotate the anchor sleeve to secure the at least one repair strand between the outer surface of the anchor sleeve and the bone socket.

The anchor tip may include a plurality of barbs. The plurality of barbs may longitudinally overlap with the channel. The anchor tip may have a tubular body that encloses at least a longitudinal portion of the channel, and the plurality of barbs may be on an outer surface of the tubular body. The tubular body may enclose an entire longitudinal length of the channel. The tubular body may have at least one proximal opening extending substantially perpendicular to a longitudinal axis of the anchor tip to allow the at least one repair strand to enter and/or exit the anchor tip in a substantially longitudinal direction. The at least one proximal opening may be formed by or may be proximal of a proximal-most barb of the plurality of barbs. The channel may have a first lateral opening and a second lateral opening, the first lateral opening and the second lateral opening extending along the longitudinal axis of the anchor tip. The plurality of barbs may include at least one barb distal of the first lateral opening and the second lateral opening and at least one barb proximal of the first lateral opening and the second lateral opening. The anchor tip may have a central wall separating the channel into a first longitudinal portion and a second longitudinal portion, the at least one repair strand may be configured to extend through the first longitudinal portion, around a distal end of the central wall, and through the second longitudinal portion. The at least longitudinal portion of the first longitudinal portion and the second longitudinal portion may be enclosed by a circumferential wall of the anchor tip. The first longitudinal portion and the second longitudinal portion may be recessed in an outer surface of the anchor tip. The plurality of barbs may include at least one distal barb distal of the first longitudinal portion and the second longitudinal portion, and at least one proximal barb circumferentially interrupted by the first longitudinal portion and the second longitudinal portion. The anchor sleeve may be proximal of the anchor tip along the drive shaft. A traction suture may be configured to releasably secure the anchor tip to a distal portion of the drive shaft. The anchor tip may have a socket, the drive shaft may have a threaded protrusion on a distal portion, and the socket may receive the threaded protrusion to releasably secure the anchor tip to the distal portion of the drive shaft. The anchor assembly may further have the at least one repair strand. The at least one repair strand may include a first repair strand and a second repair strand. The at least one repair strand may be a flat tape.

A second aspect of the present disclosure is directed to an anchor assembly configured to secure a tissue to a bone using at least one repair strand. The anchor assembly comprises an anchor tip having a tubular body with a circumferential wall, a central wall, and a channel configured to receive the at least one repair strand. The circumferential wall encloses a longitudinal portion of the channel, and the central wall separates the channel into a first longitudinal portion and a second longitudinal portion. The anchor assembly also comprises an anchor sleeve configured to be inserted into a bone socket of the bone. The anchor sleeve has an outer thread on an outer surface of the anchor sleeve. The anchor assembly also comprises a drive shaft configured to push the anchor tip into the bone socket and to rotate the anchor sleeve to secure the at least one repair strand between the outer surface of the anchor sleeve and the bone socket.

The tubular body of the anchor tip may have a plurality of barbs on an outer surface of the circumferential wall and that longitudinally overlap with the channel. The tubular body may enclose an entire longitudinal length of the channel, and the tubular body may have at least one proximal opening extending substantially perpendicular to a longitudinal axis of the anchor tip. The tubular body may enclose less than an entire longitudinal length of the channel, the channel may have a first lateral opening and a second lateral opening, and the first lateral opening and the second lateral opening may extend along the longitudinal axis of the anchor tip.

A third aspect of the present disclosure is directed to an anchor assembly configured to secure a tissue to a bone using at least one repair strand. The anchor assembly comprises an anchor tip having a channel and a central wall. The channel is configured to receive the at least one repair strand. The central wall separates the channel into a first longitudinal portion and a second longitudinal portion. The at least one repair strand is configured to extend through the first longitudinal portion, around a distal end of the central wall, and through the second longitudinal portion. The channel has a first lateral opening and a second lateral opening extending along the longitudinal axis of the anchor tip. The anchor assembly also comprises an anchor sleeve configured to be inserted into a bone socket of the bone. The anchor sleeve has an outer thread on an outer surface of the anchor sleeve. The anchor assembly also comprises a drive shaft configured to push the anchor tip into the bone socket and to rotate the anchor sleeve to secure the at least one repair strand between the outer surface of the anchor sleeve and the bone socket.

The anchor tip may have a plurality of barbs extending along a longitudinal axis, and the first lateral opening and the second lateral opening may circumferentially interrupt the plurality of barbs.

A fourth aspect is directed to an anchor assembly configured to secure a tissue to a bone using at least one repair strand, the anchor assembly comprising: an anchor tip having an eyelet and a central wall, the at least one repair strand being configured to be threaded through the eyelet and along the central wall, wherein the anchor tip has a distal portion distal of the eyelet and a proximal portion proximal of the eyelet including the central wall, the central wall has a first width, the distal end portion has a second width, and the second width is greater than the first width; an anchor sleeve configured to be inserted into a bone socket of the bone, the anchor sleeve having a lumen and an outer thread on an outer surface; and a drive shaft received in the lumen, wherein the drive shaft is configured to push the anchor tip into the bone socket and to rotate the anchor sleeve to secure the at least one repair strand between the outer surface of the anchor sleeve and the bone socket, and at least a subportion of the proximal portion is sized to be greater than the lumen to retain the at least the subportion distal of the anchor sleeve.

The central wall may have a distal end that is rounded to reduce stress concentrations on the at least one repair strand. The central wall may be recessed from an outer surface of the proximal portion of the anchor tip. The outer surface of the proximal portion of the anchor tip may have at least one barb. The distal portion may have an outer surface that includes at least one barb. The outer surface of the proximal portion of the anchor tip may be tapered. The distal portion may have a distal surface that is rounded. The central wall may have a socket configured to receive a threaded protrusion of the drive shaft. The at least the subportion of the proximal portion may include the entire proximal portion. The proximal portion may have a second subportion including a stem configured to be received in the drive shaft and sized to be received in the anchor sleeve. The stem may be an extension of the central wall and has a width less than or equal to the first width.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In the drawings:

FIG. 1 illustrates a side view of an exemplary anchor assembly according to the present disclosure.

FIG. 2 illustrates a side view of an exemplary driver of the anchor assembly of FIG. 1.

FIG. 3 illustrates an isometric view of an exemplary anchor tip of the anchor assembly of FIGS. 1 and 2 loaded with at least one repair strand.

FIG. 4 illustrates a cross-sectional view of the anchor tip of FIG. 3.

FIG. 5 illustrates a cross-sectional view of a second exemplary anchor tip of the anchor assembly of FIGS. 1-4 loaded with at least one repair strand.

FIG. 6 illustrates a perspective view of an exemplary anchor sleeve of the anchor assembly of FIGS. 1-5.

FIG. 7 illustrates a top view of the anchor sleeve of FIG. 6.

FIGS. 8-10 illustrate an exemplary method of using the anchor assembly of FIGS. 1-7.

FIG. 11 illustrates an isometric view of a third exemplary anchor tip of the anchor assembly of FIGS. 1-10 loaded with at least one repair strand.

FIG. 12 illustrates a cross-sectional view of the third anchor tip of FIG. 11.

FIG. 13 illustrates a cross-sectional view of a fourth exemplary anchor tip of the anchor assembly of FIGS. 1-10 loaded with repair strands.

FIG. 14 illustrates an isometric view of a fifth exemplary anchor tip of the anchor assembly of FIGS. 1-10 loaded with repair strands.

FIG. 15 illustrates a cross-sectional view of the fifth anchor tip of FIG. 14.

FIG. 16 illustrates a cross-sectional view of a sixth exemplary anchor tip of the anchor assembly of FIGS. 1-10 loaded with repair strands.

FIG. 17 illustrates a perspective view of a seventh exemplary anchor tip of the anchor assembly of FIGS. 1-10 loaded with repair strands.

FIG. 18 illustrates a side view of the seventh exemplary anchor tip of FIG. 17.

FIG. 19 illustrates a cross-sectional view of the seventh anchor tip of FIG. and 18.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

The present disclosure provides an anchor assembly for a knotless fixation of a tissue (e.g., a soft tissue) with improved fixation strength and/or tensioning of the suture. The anchor assembly may include an anchor tip, an anchor sleeve, and a drive shaft configured to deploy the anchor tip and the anchor sleeve. The anchor tip may secure at least one repair strand that is configured to attach to the tissue, and the anchor sleeve may have an outer thread configured to secure the anchor assembly into a socket of a bone. The drive shaft may be configured to deploy the anchor tip by pushing the anchor tip into the bone socket and be configured to deploy the anchor sleeve by rotating the anchor sleeve. The anchor tip may have a plurality of barbs configured to provide additional security to prevent pullout.

The anchor tip may have a channel configured to receive at least one repair strand and allow tensioning of the at least one repair strand to secure the tissue to the bone prior to insertion of the anchor sleeve. The anchor tip may have a central wall configured to allow the at least one repair strand to be looped through the anchor tip. The anchor assembly may be double-loaded with a first repair strand and a second repair strand. The anchor tip may have a plurality of barbs that longitudinal overlap with the channel to initially secure the anchor tip into the bone socket, while still allowing the at least one repair strand to be tensioned.

For example, the anchor tip may have a tubular body have a circumferential wall that encloses at least a longitudinal portion of the channel and allows the at least one repair strand to be tensioned with reduced engagement with the bone. In one example, the circumferential wall may enclose an entire longitudinal length of the channel. In another example, the circumferential wall may enclose less than an entire longitudinal length of the channel. The tubular body may have an open proximal portion configured to allow the at least one repair strand to enter/exit the anchor tip in a substantially longitudinal direction reducing bends in the path. Alternatively, the tubular body may have at least one lateral opening configured to allow the at least one repair strand to enter/exit the anchor tip in a lateral direction. The at least barb may be on an outer surface of the tubular body. In one example, the anchor tip may have at least one barb proximal of the at least one lateral opening to provide some degree of resistance to the suture. In another example, the channel may be recessed along an outer surface of the anchor tip. The channel may longitudinally overlap with the at least one barb to secure the anchor tip, but the channel may at least partially recess the at least one repair strand from the at least one barb to reduce the resistance to tensioning.

After the desired position of the tissue is obtained based on tensioning the at least one repair strand, the anchor assembly may be secured or set by threading the anchor sleeve into the bone socket. The at least one repair strand may be compressed between the thread of the anchor sleeve and the bone (with or without an intervening structure) to secure the at least one repair strand and prevent further translation of the at least one repair strand.

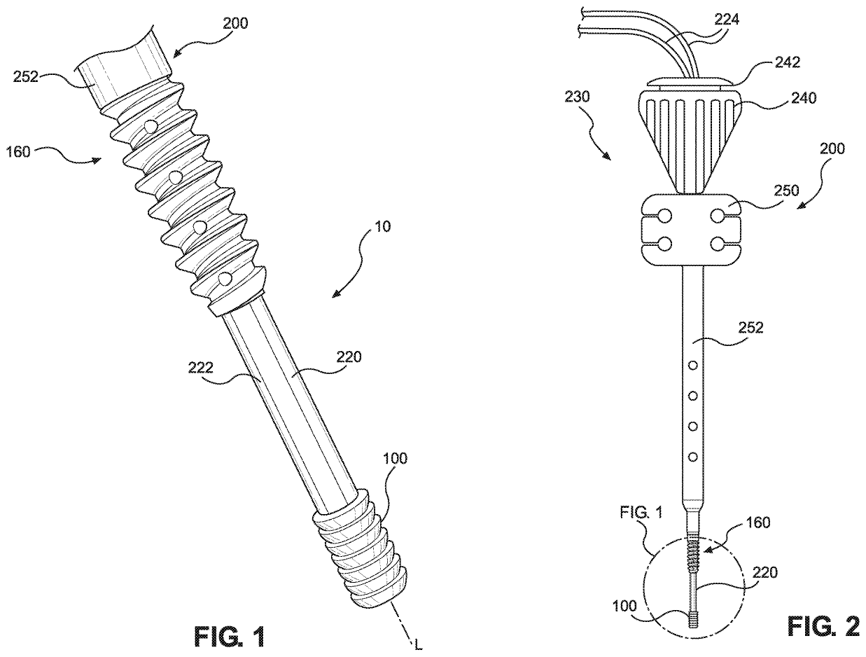

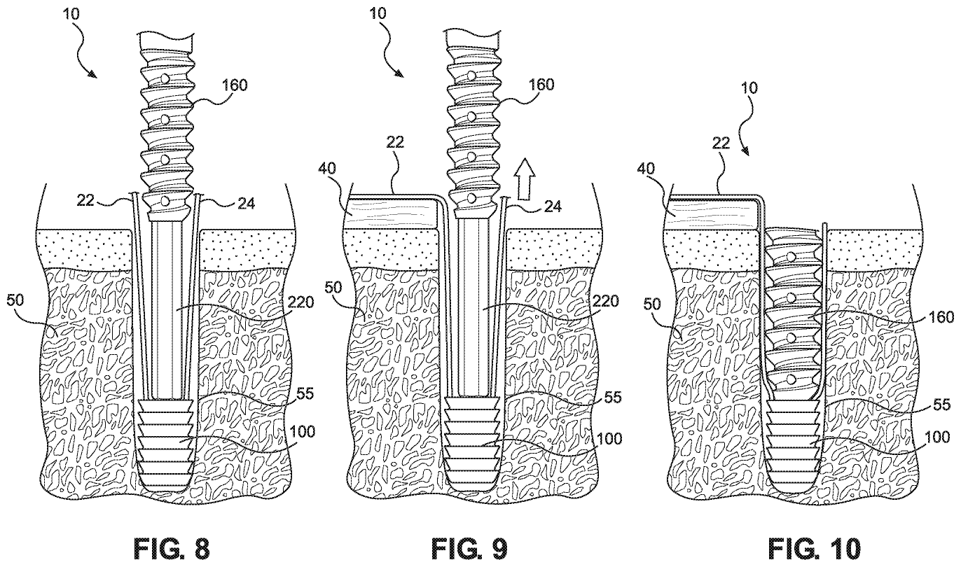

FIG. 1 illustrates an anchor assembly 10 configured to anchor a tissue 40 to a bone 50 with at least one repair strand 20, 30, as further illustrated in the method of FIGS. 8-10. The anchor assembly 10 may include an anchor tip 100 configured to receive the at least one repair strand 20, 30 and an anchor sleeve 160 configured to secure the anchor assembly 10 to the bone 50. The anchor tip 100 and the anchor sleeve 160 may be loaded onto a drive shaft 220 of a driver 200 for insertion. The drive shaft 220 may retain the anchor tip 100 and the anchor sleeve 160 along a longitudinal axis L. As further illustrated in FIG. 2, the driver 200 may have a handle 230 including a first handle member 240 attached to the drive shaft 220 and a second handle member 250 attached to a sleeve 252 disposed around the drive shaft 220.

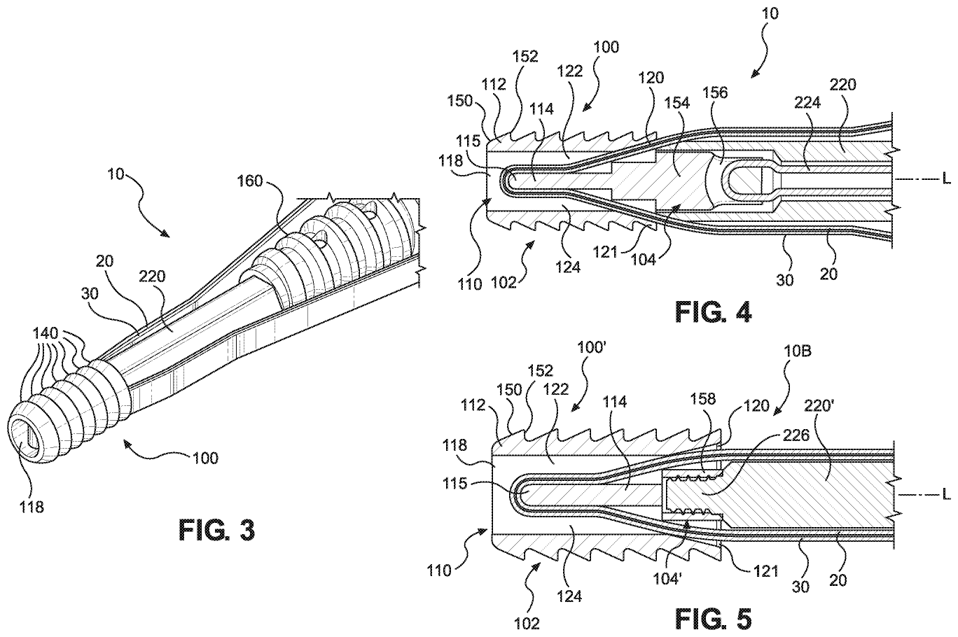

As further illustrated in FIGS. 3 and 4, the anchor tip 100 may be a separate structure from the anchor sleeve 160. In use, the anchor sleeve 160 may be configured to rotate relative to the anchor tip 100 (about the longitudinal axis L) to facilitate insertion of the anchor sleeve 160 into a bone socket 55 of the bone 50. The rotation of the anchor sleeve 160 relative to the anchor tip 100 may ensure proper orientation of the tissue 40 after being set and reduce strain on the at least one repair strand 20, 30. The anchor tip 100 may have a first portion 102 and a second portion 104. The first portion 102 may be configured to secure the at least one repair strand 20, 30 to the anchor tip 100 and/or secure the anchor tip 100 to the bone 50. The second portion 104 may be configured to removably secure the anchor tip 100 to the drive shaft 220.

The first portion 102 may have a channel 110 configured to receive the at least one repair strand 20, 30. The first portion 102 may have at least one barb 140 on an outer surface configured to secure the anchor tip 100 to the bone 50. The channel 110 may extend at least partially along the longitudinal axis L. The channel 110 may allow tensioning of the at least one repair strand 20, 30 to secure the tissue 40 to the bone 50, while the at least one barb 140 may secure the anchor tip 100 in the bone 50. The at least one barb 140 may extend at least partially around the longitudinal axis L of the anchor tip 100 and longitudinally overlap with the channel 110, as illustrated in FIGS. 3 and 4. The at least one barb 140 may include a plurality of barbs 140 longitudinally separated along the longitudinal axis L of the anchor tip 100, from a proximal portion of the anchor tip 100 to a distal portion of the anchor tip 100. As illustrated in FIG. 4, each of the barbs 140 may have a tapered distal portion 150 configured to facilitate insertion of the anchor tip 100 into the bone socket 55 and a lateral portion 152 having a greater width to provide pull-out strength from the bone socket 55. The at least one barb 140 may allow rotation of the anchor tip 100 inside of the bone socket 55 but prevent proximal translation. As further illustrated in FIGS. 3 and 4, at least some or all of the barbs 140 may extend around the entire circumference of the anchor tip 100 in an uninterrupted manner to increase pull-out strength.

The first portion 102 may have a tubular body 112 with a circumferential wall or sleeve that encloses at least a longitudinal portion of the channel 110. The tubular body 112 may enclose the at least one repair strand 20, 30 to allow the at least one repair strand 20, 30 to be tensioned with reduced engagement with the bone 50 and thus reduce resistance. The at least barb 140 may be on an outer surface of the tubular body 112. The tubular body 112 may have an open proximal portion having at least one proximal opening 120, 121. The at least one proximal opening 120, 121 may extend substantially perpendicular to the longitudinal axis L of the anchor tip 100 to allow the at least one repair strand 20, 30 to enter and/or exit the anchor tip 100 in a substantially longitudinal direction reducing bends in the path. The at least one proximal opening 120, 121 may be formed by (as illustrated in FIG. 4) or be proximal of the proximal-most barb 140 to reduce or prevent interaction of the repair strand 20, 30 with the at least one barb 140. The inner surface of the tubular body 112 at the at least one proximal opening 120, 121 may be tapered to proximally widen the channel 110 and reduce stress concentrations on the at least one repair strand 20, 30. As further illustrated in FIG. 4, the at least one repair strand 20, 30 may extend from the at least one proximal opening 120, 121 proximally along an outer surface of the drive shaft 220. The at least one proximal opening 120, 121 may have a first proximal opening 120 on a first side of the second portion 104 and a second proximal opening 121 on a second side of the second portion 104. The tubular body 112 may have a distal opening 118. The channel 110 may extend the entire length of the tubular body 112, and/or the entire length of the channel 110 may be enclosed by the tubular body 112.

The anchor tip 100 may have a central wall 114 configured to allow the at least one repair strand 20, 30 to extend or be looped through the channel 110 of the anchor tip 100 from one side to another side of the anchor tip 100 and/or drive shaft 220. The central wall 114 may extend from a first inner surface of the tubular body 112 to a second inner surface of the tubular body 112. The central wall 114 may separate the channel 110 into a first longitudinal portion 122 along a first lateral surface of the central wall 114 and a second longitudinal portion 124 along a second lateral surface of the central wall 114, providing the channel 110 with a U-shape. The at least one repair strand 20, 30 may extend through the first proximal opening 120, longitudinally through the first longitudinal portion 122, laterally around a distal end 115 of central wall 114, longitudinally through the second longitudinal portion 124, and through the second proximal opening 121. The distal end 115 of the central wall 114 may provide a hairpin turn connecting the first longitudinal portion 122 and the second longitudinal portion 124 of the channel 110, as illustrated in FIG. 4. The distal end 115 of the central wall 114 may be rounded to reduce stress concentrations on the at least one repair strand 20, 30. The anchor assembly 10 may be double-loaded with a first repair strand 20 and a second repair strand 30.

As illustrated in FIG. 4, the first portion 102 of the anchor tip 100 may have a width or diameter larger than a width or diameter of the drive shaft 220, such that the anchor tip 100 is configured to be retained at a distal end of the drive shaft 220. The channel 110 may be loaded with the at least one repair strand 20, 30 before being inserted into the bone 50. A clearance may be provided between the tubular body 112 and the tip of the drive shaft 220 for exit of the at least one repair strand 20, 30. The drive shaft 220 may contact the outer surface of the second portion 104 to apply a force and push the anchor tip 100 into the bone 50.

The second portion 104 may be releasably secured to the distal portion of the drive shaft 220. As illustrated in FIG. 4, the second portion 104 may have a stem 154 extending proximally from the first portion 102. The stem 154 may be integral to the central wall 114 and continuously extend from the central wall 114. The stem 154 may have an outer width or diameter smaller than the inner diameter of a lumen of the drive shaft 220, such that the stem 154 may be received in the lumen of the drive shaft 220 and the drive shaft 220 may apply a longitudinal pushing force to the stem 154. The stem 154 may be sized such that rotation of the drive shaft 220 does not apply torque to the anchor tip 100. The stem 154 may have an aperture 156 releasably receiving a traction suture 224 to prevent the anchor tip 100 from distally separating from the drive shaft 220 during insertion. The aperture 156 may extend transversely through the second portion 104 such that the traction suture 224 loops through the aperture 156 from one lateral side to another lateral side. As illustrated in FIG. 2, at least one (e.g., both) of the ends of the traction suture 224 may extend through the drive shaft 220 and out of a proximal opening of the driver 200 to be manipulated. The traction suture 224 may be releasably secured to the first handle member 240 to prevent inadvertent separation of the anchor tip 100 from the drive shaft 220 during insertion. For example, the traction suture 224 may be circumferentially wrapped and/or looped around the first handle member 240 and/or be received in a channel 242 to secure the traction suture 224 to the handle 230. The traction suture 224 may, additionally or alternatively, be wedged and/or secured to a cleat (not shown) on the first handle member 240. The traction suture 224 may be a round suture, a flat suture tape, an allograft, and/or a biological component. The traction suture 224 may be cored and/or coreless. After the anchor assembly 10 is implanted, the traction suture 224 may be released from the driver 200. The traction suture 224 may be subsequently used for additional tie-down of the tissue 40 (and/or other tissue) after being removed of the driver 200. For example, a suture shuttle (not shown) may be spliced into a lumen of the traction suture 224. The suture shuttle may have a loop at a distal end configured to receive an end of the traction suture 224 after the traction suture 224 is threaded through and/or around the tissue 40. The suture shuttle may then pull the traction suture 224 into itself and through the lumen to form a tensionable, knotless securement of the tissue 40 to the anchor tip 100. Further discussion of the suture shuttle and/or the knotless securement is provided in U.S. Pat. No. 9,855,029, the entire disclosure of which is expressly incorporated herein for sake of brevity.

Alternatively, as illustrated in the second anchor assembly 10B of FIG. 5, the second portion 104′ may have a socket 158 configured to receive a threaded protrusion 226 on a distal portion of the drive shaft 220′. The threads on the drive shaft 220′ may be reverse threads. Therefore, the anchor tip 100′ may be disengaged after the anchor sleeve 160 is inserted. For example, the anchor sleeve 160 may be rotated in a clockwise direction during insertion, while the drive shaft 220′ may be disengaged from the anchor tip 100′ when the drive shaft 220′ is rotated in a counterclockwise direction.

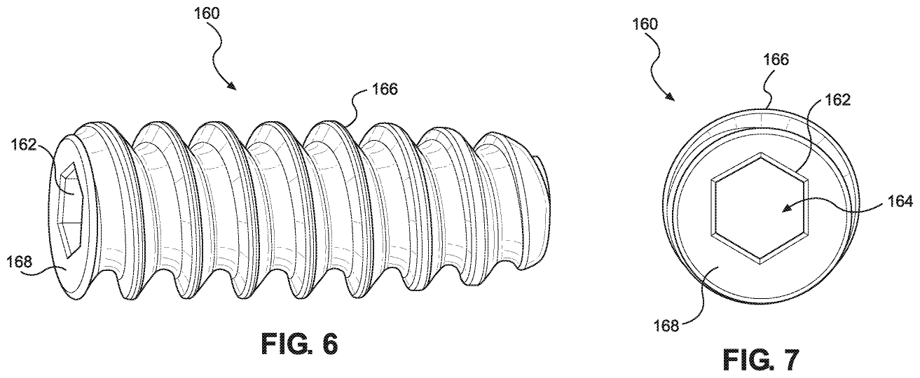

As illustrated in FIGS. 6 and 7, the anchor sleeve 160 may have an inner surface 162 defining a lumen 164 and an outer surface defining an outer thread 166. The lumen 164 of the anchor sleeve 160 may have a proximal opening through a proximal surface 168 and a distal opening through a distal surface (not shown) to receive the drive shaft 220 entirely therethrough for deployment of the anchor sleeve 160 into the bone 50. The inner surface 162 of the anchor sleeve 160 may correspond to an outer surface 222 of the drive shaft 220. A width of the inner surface 162 of the anchor sleeve 160 may be substantially equal to or greater than a width of an outer surface 222 of the drive shaft 220. The corresponding surfaces 162, 222 may allow the anchor sleeve 160 to slide relative to the drive shaft 220 during installation into the bone 50. The corresponding surfaces 162, 222 may also allow the drive shaft 220 to apply a torque to the anchor sleeve 160 during installation. Each of the surfaces 162, 222 may have a non-circular cross-section to enable the drive shaft 220 to apply the torque to the anchor sleeve 160. As illustrated, the inner surface 162 may have a hexagonal cross-section configured to mate with a corresponding hexagonal cross-section of the outer surface 222 of the drive shaft 220. Alternatively, the corresponding cross-sections of the inner surface 162 of the anchor sleeve 160 and the outer surface 222 of the drive shaft 220 may be triangular, square, pentagonal, octagonal, and/or cruciform. The second portion 104 of the anchor tip 100 may be received in the distal opening of the lumen 164 of the anchor sleeve 160 when installed in the bone socket 55.

The anchor tip 100, and/or the anchor sleeve 160 may be formed of a biocompatible and/or biosorbable material, such as a polymer, metal, and/or biological material. The anchor tip 100 and/or the anchor sleeve 160 may be formed of a bioabsorbable material, such as poly-(L-lactic acid) (PLLA), poly-(D, L-lactide), and poly glycolic acid (PGA), for example, or other bioabsorbable, non-metallic materials, which may be especially tailored for hardness, tensile strength and compressive strength. Additionally or alternatively, the anchor tip 100 and/or the anchor sleeve 160 may be formed of a metal such as titanium, titanium alloy, stainless steel or stainless steel alloy. Other biocompatible materials which may be used include plastics, allograft bone and/or inert bone substitute materials.

The at least one repair strand 20, 30 may be a high-strength suture, formed from at least one fiber of ultra-high molecular weight polyethylene (UHMWPE). The at least one repair strand 20, 30 may be a FiberWire® suture, produced by Arthrex and disclosed in U.S. Pat. No. 6,716,234, the entire disclosure of which is incorporated herein by reference. FiberWire® suture may be formed of at least one UHMWPE fiber braided with at least one other fiber to form lengths of suture material. The FiberWire® suture may include a core within a hollow braided construct, the core being a twisted yarn of UHMWPE. Additionally or alternatively, the at least one repair strand 20, 30 may include TigerWire® suture, FiberChain® suture, or TightRope® suture (all produced by Arthrex), although other cored or coreless sutures may be utilized. Additionally or alternatively, the at least one repair strand 20, 30 may be a flat tape (as illustrated in FIG. 3) made of a suture material, such as FiberTape® produced by Arthrex. Additionally or alternatively, the at least one repair strand 20, 30 may be an allograft and/or have a biological component. The allograft and/or biological component may be tendon or pericardium to provide improved tissue repair. Any combination of suture, suture tape, an allograft, and/or biological component may be employed, depending on the characteristics of the specific surgical repair and/or as desired.

Returning to FIG. 2, the first handle member 240 of the handle 230 may be attached to the drive shaft 220 and the second handle member 250 of the handle 230 may be attached to the sleeve 252 disposed around the drive shaft 220. The first handle member 240 may be proximal of the second handle member 250. The handle 230 may be manipulated to position the anchor tip 100 and the anchor sleeve 160 into the bone 50. The anchor tip 100 may be held at the distal end of the drive shaft 220 such as with the traction suture 224, and the anchor sleeve 160 may be received around the drive shaft 220 on the outer surface 222. Through manipulation of the handle 230, the drive shaft 220 may initially push the anchor tip 100 into the bone socket 55 and position the anchor sleeve 160 at the proximal opening of the bone socket 55. The drive shaft 220 may be rotated to advance the anchor sleeve 160 into the bone socket 55, for example by rotating the first handle member 240 relative to the second handle member 250. The rotation of the first handle member 240 may apply torque to the drive shaft 220 to cause rotation of the anchor sleeve 160 through engagement of the surfaces 162, 222. The second handle member 250 may be held stationary with one hand as the first handle member 240 is rotated with another hand. The engagement of the outer thread 166 to the bone socket 55 of the second tissue 40 may cause advancement of the anchor sleeve 160 into the bone 50. Further discussion of the anchor tip 100, the anchor sleeve 160, and the driver 200 is provided in U.S. Pat. No. 10,820,897, the entire disclosure of which is incorporated herein by reference.

FIGS. 8-10 illustrates a method of using the anchor assembly 10. The method may be used to secure a tissue 40 to a bone 50 with the at least one repair strand 20, 30 (only a single repair strand 20 being illustrated for simplicity). The tissue 40 may be a soft tissue or bone. For example, the method may be for repairing a torn rotator cuff by attaching the tissue 40 to its original site on the bone 50 and in its original orientation. Thus, the tissue 40 may be a supraspinatus tendon, and the bone 50 may be a humerus of a human shoulder. However, the method may have a variety of other applications in securing tissue 40 to bone 50. For example, the tissue 40 may be a bone such as a talus, and the bone 50 may be a fibula in performing AC joint stabilization.

As illustrated in FIG. 8, the bone socket 55 may be formed into the bone 50. The bone socket 55 may be formed by a punch. The anchor tip 100 may be inserted with the driver 200 into the bone socket 55, with the drive shaft 220 pushing the anchor tip 100 and the traction suture 224 holding the anchor tip 100 against the drive shaft 220. The driver 200 may position the anchor tip 100 at the bottom of the bone socket 55 or otherwise at a desired depth.

The at least one repair strand 20, 30 may be preloaded into the anchor tip 100. Alternatively, the at least one repair strand 20, 30 may be shuttled through the anchor tip 100 with a suture threader after being secured to the bone 50 with another anchor. The at least one barb 140 may frictionally secure the anchor tip 100 in the bone socket 55. Ends 22, 24 of each of the at least one repair strand 20, 30 may extend from the bone socket 55 to be manipulated by the user. The ends 22, 24 may be free to translate through the channel 110 of the anchor tip 100 for manipulation by the user.

As illustrated in FIG. 9, a first end 22 may be secured to the tissue 40. For example, the first end 22 of the repair strand 20 may be looped through the tissue 40 with a suture passer, such as that described in U.S. Pat. No. 7,972,344 as incorporated herein by reference in its entirety. As illustrated, the first end 22 of the repair strand 20 may be secured to the tissue 40 by being tied into a knot with the remaining length of the first end 22 being cut. The second end 24 may be pulled to position the tissue 40 in the desired position and orientation for repair on the outer surface of the bone 50. The tensioning of the repair strand 20 may secure the tissue 40 directly against the bone 50, enhancing the footprint of compression and accelerating healing of the tissue 40. Alternatively, the repair strand 20 may be attached to the soft tissue prior to being threaded through the anchor tip 100. The driver 200 may initially position the distal end of the anchor sleeve 160 outside of the bone socket 55 at or near the proximal opening of the bone socket 55 with the anchor tip 100 positioned in the bone socket 55.

As illustrated in FIG. 10, the anchor sleeve 160 may be advanced into the bone socket 55. The anchor sleeve 160 may be advanced by rotating the drive shaft 220. The outer thread 166 of the anchor sleeve 160 may engage an inner wall of the bone socket 55 or an intervening structure such as an additional, peripheral anchor sleeve. The anchor sleeve 160 may advance along the drive shaft 220 toward the anchor tip 100, as the drive shaft 220 rotates the anchor sleeve 160. The anchor sleeve 160 may secure the tension in the repair strand 20, 30 through compression of the repair strand 20 between the anchor sleeve 160 and the bone socket 55, such as directly against the wall of the bone socket 55 or against an intervening structure such as an additional anchor sleeve. At least a portion of the anchor tip 100 may have a width greater than a width of the lumen 164 of the anchor sleeve 160 to retain the anchor tip distal of the anchor sleeve 160. Tension applied to the tissue 40 by the repair strand 20 may be evaluated. The tension on the repair strand 20, 30 may be adjusted by backing out the anchor sleeve 160 with the driver 200, adjustment of the repair strand 20, 30, and reentry of the anchor sleeve 160 to fix the repair strand 20, 30. After the desired tension is achieved by the anchor assembly 10, the traction suture 224 may be released from the driver 200. The drive shaft 220 may be detached from the anchor tip 100 and proximally withdrawn from the anchor sleeve 160. The second end 24 of the repair strand 20 may be trimmed after final placement of the anchor assembly 10 with the repair strand 20 compressed between the anchor sleeve 160 and the bone 50. The second repair strand 30 may be used to secure the same or different tissue 40.

Additional bone sockets 55 may be formed, and additional anchor assemblies 10 may be inserted into the additional bone sockets 55. For example, the method may be performed in a single-or a double-row repair of the rotator cuff, such as a double-row suture bridging technique as described in U.S. Pat. No. 8,419,794, as incorporated herein by reference in its entirety.

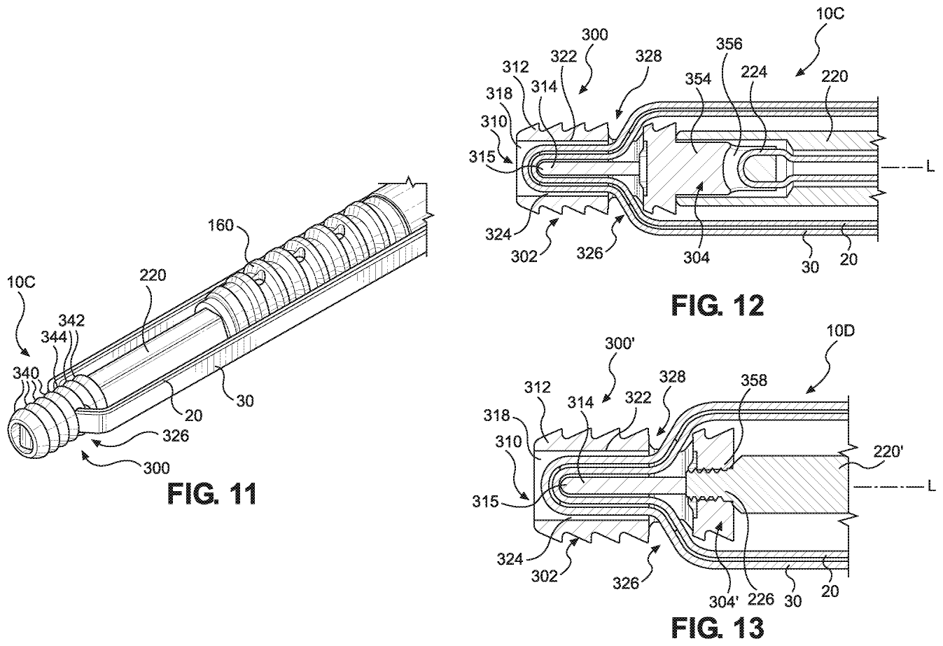

FIGS. 11 and 12 illustrate a third anchor assembly 10C. The third anchor assembly 10C may have similar features as the first anchor assembly 10, the entire disclosure of which is incorporated by reference for sake of brevity except when otherwise indicated. As illustrated, the third anchor assembly 10C may have an anchor tip 300 with a first portion 302 and a second portion 304. The first portion 302 may be configured to secure the at least one repair strand 20, 30 to the anchor tip 300 and/or secure the anchor tip 300 to the bone socket 55. The second portion 304 may be configured to removably secure the anchor tip 300 to the drive shaft 220.

The first portion 302 may have a channel 310 configured to receive the at least one repair strand 20, 30 and at least one barb 340, 342, 344 configured to secure the anchor tip 300 to the bone 50. The channel 310 may extend at least partially along the longitudinal axis L. The channel 310 may allow tensioning of the at least one repair strand 20, 30 to secure the tissue 40 to the bone 50, while the at least one barb 340 secures the anchor tip 300 in the bone 50. The at least one barb 340, 342, 344 may extend at least partially around the longitudinal axis L of the anchor tip 300 and longitudinally overlap with the channel 310. The at least one barb 340, 342, 344 may include a plurality of barbs 340, 342, 344 longitudinally separated along the longitudinal axis L of the anchor tip 300, from a proximal portion of the anchor tip 300 to a distal portion of the anchor tip 300.

The first portion 302 may have a tubular body 312 with a circumferential wall or sleeve that encloses a longitudinal portion of the channel 310. The tubular body 312 may enclose less than an entire longitudinal length and have lateral openings 326, 328. For example, the tubular body 312 may enclose greater than half of a longitudinal length of the channel 310. The anchor tip 300 may have a central wall 314 configured to allow the at least one repair strand 20, 30 to extend or be looped through the channel 310 of the anchor tip 300 from one side to another side of the anchor tip 300 and/or drive shaft 220. The central wall 314 may extend from a first inner surface of the tubular body 312 to a second inner surface of the tubular body 312 separating the channel 310 into a first longitudinal portion 322 and a second longitudinal portion 324. The channel 310 of the anchor tip 300 may further have a first lateral opening 326 and a second lateral opening 328, with the first lateral opening 326 in communication with and formed by the first longitudinal portion 322 and the second lateral opening 328 in communication with and formed by the second longitudinal portion 324. The lateral openings 326, 328 may extend through an outer surface of the anchor tip 300 along the longitudinal axis L. The at least one repair strand 20, 30 may extend laterally through the first lateral opening 326, longitudinally through the first longitudinal portion 322, laterally around a distal end 315 of central wall 314, longitudinally through the second longitudinal portion 324, and laterally through the second lateral opening 328. The distal end 315 of the central wall 314 may be rounded to reduce stress concentrations on the at least one repair strand 20, 30. The tubular body 312 may have a distal opening 318.

The anchor tip 300 may have at least one distal barb 340 distal of the lateral openings 326, 328, at least one proximal barb 342 proximal of the lateral openings 326, 328, and at least one middle barb 344 that may be circumferentially interrupted by the lateral openings 326, 328. The at least one distal barb 340 and/or proximal barb 342 may extend entirely around the circumference of the anchor tip 300, and the at least one middle barb 344 may extend partially around the circumference of the anchor tip 300 being circumferentially interrupted by the lateral openings 326, 328. As illustrated, the at least one repair strand 20, 30 may extend laterally through the lateral openings 326, 328 and engage the proximal barbs 342 providing some degree of resistance to the at least one repair strand 20, 30. However, as illustrated, the anchor tip 300 may have greater number distal barbs 340 than proximal barbs 342 reducing the resistance. For example, as illustrated, the anchor tip 300 may have four distal barbs 340 and two proximal barbs 342. Alternatively, the proximal barbs 342 may extend partially around the circumference of the anchor tip 300 and be circumferentially interrupted by the lateral openings 326, 328 forming recessed first and second longitudinal portions of the channel 310 to reduce resistance of the at least one repair strand 20, 30, as illustrated and discussed with reference to FIGS. 15-16.

As illustrated in FIG. 12, the second portion 304 may have a stem 354 extending proximally from the first portion 302. The second portion 304 have an aperture 356 releasably receiving a traction suture 224 to prevent the anchor tip 100 from distally separating from the drive shaft 220 during insertion, similar to the first anchor assembly 10, the entire disclosure of which is incorporated herein by reference. Alternatively, as illustrated in FIG. 13, the second portion 304′ may have a socket 358 configured to receive a threaded protrusion 226 on a distal portion of the drive shaft 220′, similar to the second anchor assembly 10B, the entire disclosure of which is incorporated herein by reference.

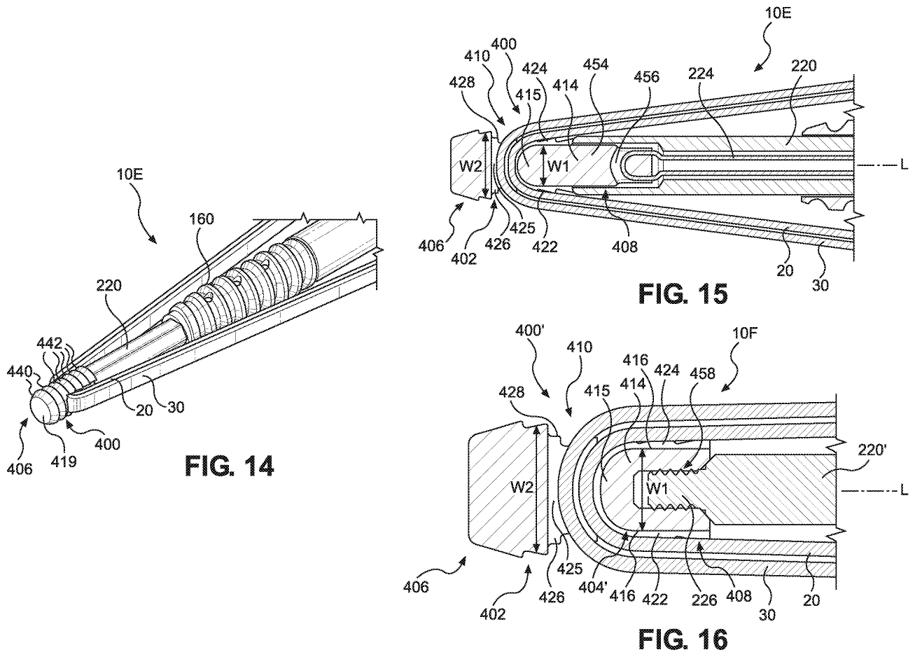

FIGS. 14 and 15 illustrate a fifth anchor assembly 10E. The fifth anchor assembly 10E may have similar features as at least one of the first through fourth anchor assemblies 10A-D, the entire disclosure of which is incorporated by reference for sake of brevity except when otherwise indicated. As illustrated, the fifth anchor assembly 10E may have an anchor tip 400 configured to secure the at least one repair strand 20, 30 to the anchor assembly 10E. The anchor tip 400 may have a channel 410 configured to receive the at least one repair strand 20, 30 and at least one barb 440, 442 on an outer surface configured to secure the anchor tip 400 to the bone 50.

The channel 410 may include an eyelet 425 extending laterally through the anchor tip 400, wherein the at least one repair strand 20, 30 is configured to be looped through the eyelet 425. The anchor tip 400 may have a distal portion 406 distal of the eyelet 425 and a proximal portion 408 proximal of the eyelet 425. The proximal portion 408 may include a central wall 414, wherein the at least one repair strand 20, 30 may be threaded through the eyelet 425 and be received on opposing sides of the central wall 414. The eyelet 425 may laterally extend between a first lateral opening 426 and a second lateral opening 428. The distal portion 406 may define a distal extent of the eyelet 425, and the central wall 414 may define a proximal extent of the eyelet 425. A proximal surface and/or a distal surface of the distal portion 406 may be rounded and/or flat. The distal end 415 of the central wall 414 may be rounded at the proximal extent of the eyelet 425 to reduce stress concentrations on the at least one repair strand 20, 30 looped through the eyelet 425. The central wall 414 may have substantially flat or planar surfaces 416 extending proximally from the distal end 415. The substantially flat or planar surfaces 416 may allow for flush abutment of flat repair strands 20, 30. The surfaces 416 may be substantially parallel and run along the longitudinal axis L of the anchor tip 400. The central wall 414 may have a first width W1, for example defined by the distance between the surfaces 416 proximal of the rounded distal end 415. The central wall 414 defining the first width W1 may extend to the proximal end of the anchor tip 400. The distal portion 406 may have a second width W2, where the second width W2 is greater than the first width W1. At least a portion of the central wall 414 may be recessed from the outer surface of the proximal portion 408, such that the outer surface of the proximal portion 408 may have a width greater than the first width W1.

The at least one repair strand 20, 30 may be looped through the eyelet 425 from one side to another side of the anchor tip 400 and/or drive shaft 220. The channel 410 may extend at least partially along the longitudinal axis L. The channel 410 may allow tensioning of the at least one repair strand 20, 30 to secure the tissue 40 to the bone 50, while the at least one barb 440 secures the anchor tip 100 in the bone 50. The central wall 414 may separate the channel 410 into a first longitudinal portion 422 and a second longitudinal portion 424 on opposites sides of the eyelet 425. The longitudinal portions 422, 424 may be recessed from the outer surface of the anchor tip 400 such that the longitudinal portions 422, 424 are not radially enclosed. The first lateral opening 426 may be in communication with and extend along the first longitudinal portion 422 and the second lateral opening 428 may be in communication with and extend along the second longitudinal portion 424. The lateral openings 426, 428 may extend through the outer surface of the anchor tip 400 along the longitudinal axis L. The at least one repair strand 20, 30 may extend laterally through the first lateral opening 426, longitudinally along the first longitudinal portion 422, laterally through the eyelet 425 around a distal end 415 of central wall 414, longitudinally along the second longitudinal portion 424, and laterally through the second lateral opening 428.

The at least one barb 440, 442 may extend at least partially around the longitudinal axis L of the anchor tip 400 and longitudinally overlap with the channel 410. The at least one barb 440, 442 may include a plurality of barbs 440, 442 longitudinally separated along the longitudinal axis L of the anchor tip 400, from a proximal end of the anchor tip 400 to a distal end of the anchor tip 400. The outer surface of the anchor tip 400 may have at least one distal barb 440 on the distal portion 406 distal of the lateral openings 426, 428, and at least one proximal barb 442 on the proximal portion 408 proximal of the eyelet 425 and/or longitudinally overlapping with the recessed longitudinal portions 422, 424. The at least one distal barb 440 may extend entirely around the circumference of the anchor tip 400, and the at least one proximal barb 444 may extend partially around the circumference of the anchor tip 400 being circumferentially interrupted by the lateral openings 426, 428 and/or the recessed longitudinal portions 422, 424. The at least one repair strand 20, 30 may slide through the eyelet 425 and along the recessed longitudinal portions 422, 424 as the barbs 440, 442 secure the anchor tip 400 in the bone 50.

At least a first subportion of the proximal portion 408 may be sized to be greater than the lumen 164 of the anchor sleeve 160 to retain the at least the first subportion distal of the anchor sleeve 160. The at least the first subportion may include at least a portion of the central wall 414 and the barbs 440, 442. For example, the barbs 440, 442 may have a width greater than a width of the lumen 164 of the anchor sleeve 160. As illustrated in FIG. 15, the proximal portion 408 may include a second subportion including a stem 454 extending proximally of the outer surface. The stem 454 may have an aperture 456 releasably receiving a traction suture 224 to prevent the anchor tip 100 from distally separating from the drive shaft 220 during insertion. The stem 454 may extend proximally of the barbs 440, 442 and have a size less than the first subportion including the barbs 440, 442. The stem 454 may have a width less than the lumen 164 of the anchor sleeve 160, such that the stem 454 is sized to be received in the lumen 164 and a distal end of the anchor sleeve 160 may abut a proximal end of a proximal most barb 440, 442. As further illustrated in FIG. 15, the stem 454 may be an extension of the central wall 414 and have a width equal to or less than the first width (W1) of the central wall 414. The traction suture 224 may pull the stem 454 into the lumen 164 of the anchor sleeve 160, similar to the first anchor assembly 10 and/or third anchor assembly 10C, the entire disclosure of which is incorporated herein by reference. Alternatively, the first subportion may include an entirety of the proximal portion 408. For example, as illustrated in FIG. 16, the proximal portion 408′ may have a socket 458 configured to receive a threaded protrusion 226 on a distal portion of the drive shaft 220′. The socket 458 may be in a proximal end of the central wall 414. The barbs 440, 442 may extend longitudinally to a proximal end of the central wall 414, such that the proximal end of the proximal portion 408′ is larger than the lumen 164 of the anchor sleeve 160 and not sized to be received in the lumen 164 of the anchor sleeve 160. The threaded protrusion 226 on a distal portion of the drive shaft 220′ may be configured to deploy the anchor tip 400′ similar to the second anchor assembly 10B and/or fourth anchor assembly 10D, the entire disclosure of which is incorporated herein by reference.

FIGS. 17-19 illustrate a seventh anchor assembly 10G. The seventh anchor assembly 10G may have similar features as at least one of the first through sixth anchor assemblies 10A-F, the entire disclosure of which is incorporated by reference for sake of brevity except when otherwise indicated. As illustrated, the seventh anchor assembly 10G may have an anchor tip 500 configured to secure the at least one repair strand 20, 30 to the anchor assembly 10G.

The anchor tip 500 may have a channel 510 configured to receive the at least one repair strand 20, 30. The channel 510 may include an eyelet 525 extending laterally through the anchor tip 500, wherein the at least one repair strand 20, 30 is configured to be looped through the eyelet 525. The anchor tip 500 may have a distal portion 506 distal of the eyelet 525 and a proximal portion 508 proximal of the eyelet 525. The proximal portion 508 may include a central wall 514, wherein the at least one repair strand 20, 30 may be threaded through the eyelet 525 and on opposing sides of the central wall 514. The eyelet 525 may laterally extend between a first lateral opening 526 and a second lateral opening 528. The distal portion 506 may define a distal extent of the eyelet 525, and the central wall 514 may define a proximal extent of the eyelet 525. A proximal surface and/or a distal surface of the distal portion 406 may be rounded and/or flat. The distal end 515 of the central wall 514 may be rounded at the proximal extent of the eyelet 525 to reduce stress concentrations on the at least one repair strand 20, 30 looped through the eyelet 525. The central wall 514 may have substantially flat or planar surfaces 516 extending proximally from the distal end 515. The substantially flat or planar surfaces 516 may allow for flush abutment of flat repair strands 20, 30. The surfaces 516 may be substantially parallel and run along the longitudinal axis L of the anchor tip 500. The central wall 514 may have a first width W1, for example defined by the distance between the surfaces 516 proximal of the rounded distal end 515. The central wall 514 having the first width W1 may extend to the proximal end of the anchor tip 500. The distal portion 506 may have a second width W2, where the second width W2 is greater than the first width W1. At least a portion of the central wall 514 may be recessed from an outer surface 517 of the proximal portion 508.

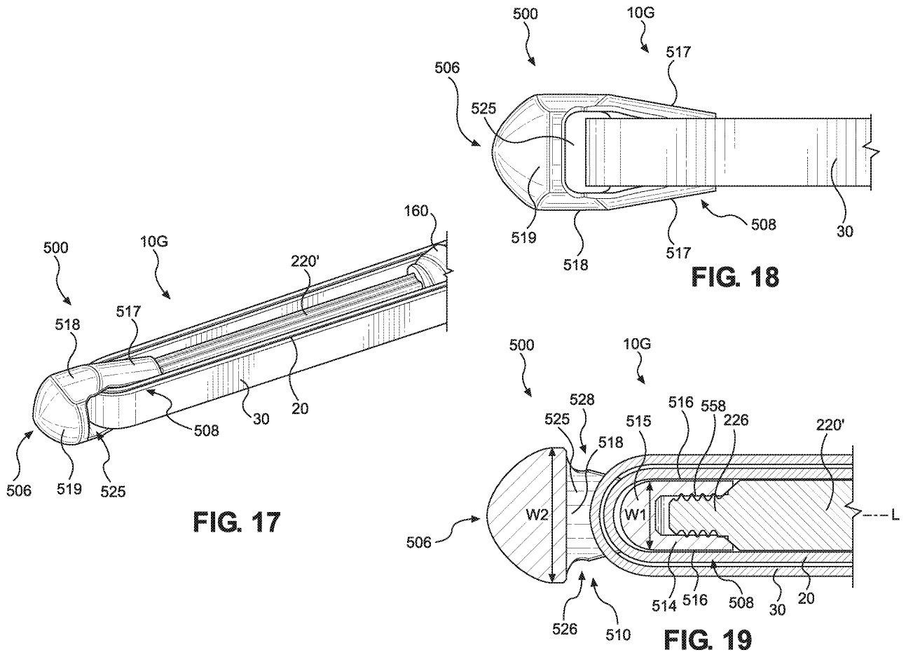

The at least one repair strand 20, 30 may be looped through the eyelet 525 from one side to another side of the anchor tip 500 and/or drive shaft 220. The anchor tip 500 may be barbless. At least a portion of the surfaces 516 of the central wall 514 may be recessed from the outer surface 517 of the proximal portion 508, such that the outer surface of the proximal portion 508 may have a width greater than the first width W1. The outer surface 517 may be on opposing sides of each of the surfaces 516 of the central wall 514. The outer surface 517 may be rounded along a circumference of the anchor tip 500 to avoid sharp edges, as illustrated. The outer surface 517 may be tapered and extend at an acute angle relative to the longitudinal axis L of the anchor tip 500, as illustrated in FIGS. 17 and 18. The outer surface 517 may be proximally tapered from a larger distal width to a smaller proximal width. For example as illustrated, the smaller proximal width may be on a proximal end of a first subportion of the proximal portion 508 or the entirety of the proximal portion 508. Alternatively, the outer surface 517 may not be tapered and extend along the longitudinal axis L with a substantially uniform cross-section along its length. The distal portion 506 may have a rounded distal surface 519 to facilitate insertion into the bone socket 55. A medial portion 518 may extend between the proximal portion 508 and the distal portion 506. The medial portion 518 may at least partially form the eyelet 525. The medial portion 518 may connected the outer surface 517 of the proximal portion 508 and the distal surface 519 of the distal portion 506. The medial portion 518 may have substantially a uniform width, be rounded, or be tapered along the longitudinal axis L at a different angle than the outer surfaces 517. The medial portion 518 may be rounded along a circumference of the anchor tip 500 to avoid sharp edges, as further illustrated. The medial portion 518 may have a width larger than a width of the proximal portion 508 (e.g., defined between the outer surfaces 517), as illustrated in FIG. 18. Additionally or alternatively, the medial portion 518 may have a width substantially the same as the width defined between the outer surfaces 517 of the proximal portion 508.

At least the first subportion of the proximal portion 508 may be sized to be greater than the lumen 164 of the anchor sleeve 160 to retain the at least the first subportion distal of the anchor sleeve 160. The at least the first subportion may include at least a portion of the central wall 514 and the outer surface 517. The proximal portion 508 may include a second subportion including a stem (as illustrated for example in FIG. 15) extending proximally of the outer surface 517. The stem may have an aperture releasably receiving a traction suture 224 to prevent the anchor tip 100 from distally separating from the drive shaft 220 during insertion. The stem may extend proximally of the outer surface 517 and have a size less than the first subportion including the outer surface 517. The stem may be configured to be received in the drive shaft 220 and sized to be received in the lumen 164 of the anchor sleeve 160, such that a distal end of the anchor sleeve 160 may abut a proximal end of a proximal surface of the outer surface 517. The stem may be an extension of the central wall 514 and have the same width (W1) as the central wall 514. The traction suture 224 may pull the stem into the lumen 164 of the anchor sleeve 160, similar to the first anchor assembly 10, third anchor assembly 10C, and/or fifth anchor assembly 10F, the entire disclosure of which is incorporated herein by reference. Alternatively, the first subportion may include an entirety of the proximal portion 508. For example, as illustrated in FIG. 19, the proximal portion 408 may have a socket 558 configured to receive a threaded protrusion 226 on a distal portion of the drive shaft 220′. The socket 558 may be in a proximal end of the central wall 514. The outer surface 517 may extend longitudinally to a proximal end of the central wall 514, such that the proximal end of the proximal portion 508′ is larger than the lumen 164 of the anchor sleeve 160 and not sized to be received in the lumen 164 of the anchor sleeve 160. The threaded protrusion 226 on a distal portion of the drive shaft 220′ may be configured to deploy the anchor tip 300′ similar to the second anchor assembly 10B, fourth anchor assembly 10D, and/or sixth anchor assembly 10F, the entire disclosure of which is incorporated herein by reference.

It will also be appreciated by those skilled in the art that modifications can be made to the example embodiments described herein without departing from the invention. Structural features of systems and apparatuses described herein can be replaced with functionally equivalent parts or omitted entirely. Moreover, it will be appreciated that features from the embodiments can be combined with each other without departing from the disclosure.

What is claimed is:

1. An anchor assembly configured to secure a tissue to a bone using at least one repair strand, the anchor assembly comprising:
an anchor tip having a channel extending along a longitudinal axis, wherein the channel is configured to receive the at least one repair strand;
an anchor sleeve configured to be inserted into a bone socket of the bone, the anchor sleeve having an outer thread on an outer surface; and
a drive shaft configured to push the anchor tip into the bone socket and to rotate the anchor sleeve to secure the at least one repair strand between the outer surface of the anchor sleeve and the bone socket.

2. The anchor assembly of claim 1, wherein the anchor tip comprises a plurality of barbs.

3. The anchor assembly of claim 2, wherein the anchor tip has a tubular body that encloses at least a longitudinal portion of the channel, and the plurality of barbs are on an outer surface of the tubular body.

4. The anchor assembly of claim 3, wherein the tubular body has at least one proximal opening extending substantially perpendicular to a longitudinal axis of the anchor tip to allow the at least one repair strand to enter and/or exit the anchor tip in a substantially longitudinal direction.

5. The anchor assembly of claim 4, wherein the at least one proximal opening is formed by or is proximal of a proximal-most barb of the plurality of barbs.

6. The anchor assembly of claim 3, wherein the channel has a first lateral opening and a second lateral opening, the first lateral opening and the second lateral opening extending along the longitudinal axis of the anchor tip.

7. The anchor assembly of claim 6, wherein the plurality of barbs includes at least one barb distal of the first lateral opening and the second lateral opening and at least one barb proximal of the first lateral opening and the second lateral opening.

8. The anchor assembly of claim 1, wherein the anchor tip has a central wall separating the channel into a first longitudinal portion and a second longitudinal portion, the at least one repair strand configured to extend through the first longitudinal portion, around a distal end of the central wall, and through the second longitudinal portion.

9. The anchor assembly of claim 8, wherein at least a longitudinal portion of the first longitudinal portion and the second longitudinal portion are enclosed by a circumferential wall of the anchor tip.

10. The anchor assembly of claim 8, wherein the first longitudinal portion and the second longitudinal portion are recessed in an outer surface of the anchor tip.

11. The anchor assembly of claim 1, wherein the anchor sleeve is proximal of the anchor tip along the drive shaft.

12. The anchor assembly of claim 1, further comprising a traction suture configured to releasably secure the anchor tip to a distal portion of the drive shaft.

13. The anchor assembly of claim 1, the anchor tip has a socket, the drive shaft has a threaded protrusion on a distal portion, and the socket receives the threaded protrusion to releasably secure the anchor tip to the distal portion of the drive shaft.

14. The anchor assembly of claim 1, further comprising the at least one repair strand.

15. An anchor assembly configured to secure a tissue to a bone using at least one repair strand, the anchor assembly comprising:
an anchor tip having a tubular body with a circumferential wall, a central wall, and a channel configured to receive the at least one repair strand, the circumferential wall enclosing a longitudinal portion of the channel, and the central wall separating the channel into a first longitudinal portion and a second longitudinal portion,
an anchor sleeve configured to be inserted into a bone socket of the bone, the anchor sleeve having an outer thread on an outer surface of the anchor sleeve; and
a drive shaft configured to push the anchor tip into the bone socket and to rotate the anchor sleeve to secure the at least one repair strand between the outer surface of the anchor sleeve and the bone socket.

16. The anchor assembly of claim 15, wherein the tubular body of the anchor tip has a plurality of barbs on an outer surface of the circumferential wall and that longitudinally overlap with the channel.

17. An anchor assembly configured to secure a tissue to a bone using at least one repair strand, the anchor assembly comprising:
an anchor tip having an eyelet and a central wall, the at least one repair strand being configured to be threaded through the eyelet and along the central wall, wherein the anchor tip has a distal portion distal of the eyelet and a proximal portion proximal of the eyelet including the central wall, the central wall has a first width, the distal portion has a second width, and the second width is greater than the first width;
an anchor sleeve configured to be inserted into a bone socket of the bone, the anchor sleeve having a lumen and an outer thread on an outer surface; and
a drive shaft received in the lumen,
wherein the drive shaft is configured to push the anchor tip into the bone socket and to rotate the anchor sleeve to secure the at least one repair strand between the outer surface of the anchor sleeve and the bone socket, and at least a subportion of the proximal portion is sized to be greater than the lumen to retain the at least the subportion distal of the anchor sleeve.

18. The anchor assembly of claim 17, wherein the central wall has a distal end that is rounded to reduce stress concentrations on the at least one repair strand.

19. The anchor assembly of claim 17, wherein the central wall is recessed from an outer surface of the proximal portion of the anchor tip.

20. The anchor assembly of claim 19, wherein the outer surface of the proximal portion of the anchor tip has at least one barb.
